(19) United States
(12) Patent Application Publication
(10) Pub. No.: US 2009/0053199 A1
(43) Pub. Date: Feb. 26, 2009

(54) Stable Dairy Components Effective for Fat Loss

(76) Inventor: Michael B. Zemel, Knoxville, TN (US)

Correspondence Address: VENABLE LLP, P.O. BOX 34385, WASHINGTON, DC, 20043-9998, US

(21) Appl. No.: 11/918,277

(22) PCT Filed: Apr. 11, 2006
(86) PCT No.: PCT/US06/13454

§ 371 Date: Oct. 11, 2007

Related U.S. Application Data
(60) Provisional application No. 60/669,882, filed on Apr. 11, 2005.

(51) Int. Cl. A61K 35/20; A61P 3/04; A61K 38/38; A61K 39/395; A61K 38/40; A61K 38/44; A61P 3/00; A61P 35/00; A61P 9/12

(52) U.S. Cl. 424/130.1; 424/535; 514/12; 424/94.4

(57) ABSTRACT

The present invention relates to methods comprising administering to individuals regulating body weight calcium-containing fresh whey products in an amount effective to manage body mass, by inducing weight and/or fat loss, preventing weight and/or fat gain, increasing the metabolic consumption of adipose tissue, and/or maintaining muscle mass.

### FIELD OF THE INVENTION

The present invention relates to use of fresh or stable whey products in weight management, e.g., a method comprising administering to an individual regulating body weight an amount of a calcium-containing fresh whey product effective to induce weight loss, prevent weight gain, increase the metabolic consumption of adipose tissue, and/or maintain muscle mass on a calorie restricted diet.

### BACKGROUND OF THE INVENTION

Dietary calcium has been shown to induce weight loss, prevent weight gain and/or increase the metabolic consumption of adipose tissue. See e.g., commonly owned U.S. Pat. No. 6,384,087, Zemel, et. al., and U.S. Published Patent Application Nos. 2004-0197425, 2004-0197423, 2004-0197385, 2004-0197383, 2004-0197382, 2002-0132014, 2002-0192264, and 2004-0197424, all in the name of Zemel, et al., and which are all incorporated herein by reference in their entirety. It was not previously recognized that such effects can be achieved and enhanced by administering fresh whey products.

Individuals often gain 1-3 pounds of weight a year and when dieting, although fat loss may be achieved, it is often accompanied with loss of lean or muscle mass. There is a need for providing diets that selectively target specific fat zones while maintaining lean or muscle mass.

Also, it is difficult for many people to maintain a healthy diet including sufficient amounts of calcium and in particular dairy calcium. Consuming several portions (two to four) daily of dairy products such as milk, cheese, or yoghurt is difficult or undesirable for some people. It would be desirable to employ a dietary component that can consistently provide the weight management benefits of milk in a form that is suitable for adding to foods and formulations to fortify them.

The present invention is directed to products containing ingredients from whey that help augment weight loss while protecting muscle. Individuals consuming such products lose more fat and less lean. The invention also helps the body avoid gaining fat mass.

### SUMMARY OF THE INVENTION

The present invention relates to, e.g., methods comprising administering to an individual regulating body weight a calcium containing fresh whey product in an amount effective to induce weight and/or fat loss, prevent weight and/or fat gain, increase the metabolic consumption of adipose tissue in the individual, and/or maintain muscle mass.

### BRIEF DESCRIPTION OF THE DRAWINGS

FIG. 1 is a schematic illustration of the effects and mechanism of fresh whey products. An insufficiency of dietary calcium increases intracellular calcium ([Ca²⁺]ᵢ) in human adipocytes, thereby stimulating lipogenesis and inhibiting lipolysis, and increasing triglyceride accumulation. In contrast, increasing dietary calcium with dairy such as fresh whey products suppresses calcitrophic hormones, 1α,25-(OH)₂-D₃, thereby reducing [Ca^{2+]}ᵢ and, consequently, triglyceride accumulation.

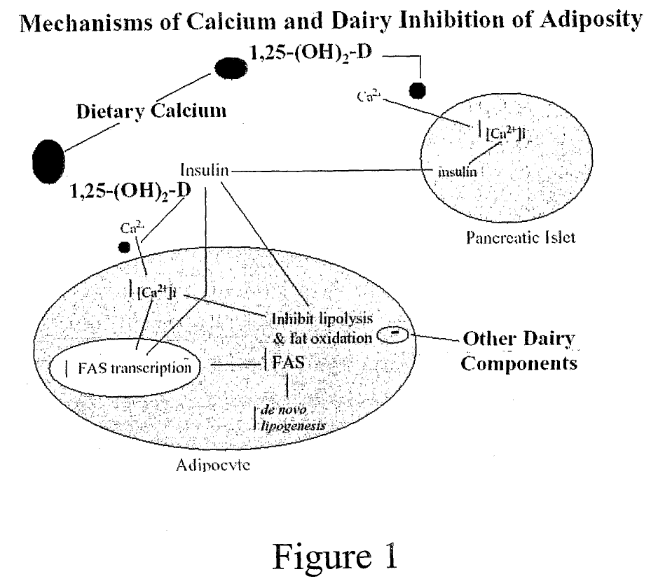

FIG. 2 outlines the specific feeding pattern of calcium and selective dairy components in eight groups oftransgenic mice expressing agouti specifically in adipocytes under the control of the aP2 promoter.

FIG. 3 shows the effects of consuming calcium and selective dairy components for six weeks on weight change in transgenic mice expressing agouti specifically in adipocytes under the control of the aP2 promoter.

FIG. 4 illustrates the effects of consuming calcium and selective dairy components for six weeks on Fat Pad Mass in transgenic mice expressing agouti specifically in adipocytes under the control of the aP2 promoter.

FIG. 5 depicts the effects of consuming calcium and selective dairy components for six weeks on Abdominal Fat in transgenic mice expressing agouti specifically in adipocytes under the control of the aP2 promoter.

FIG. 6 shows the effects of consuming calcium and selective dairy components for six weeks on Perirenal Fat in transgenic mice expressing agouti specifically in adipocytes under the control of the aP2 promoter.

FIG. 7 illustrates the effects of consuming calcium and selective dairy components for six weeks on Subscapular Fat in transgenic mice expressing agouti specifically in adipocytes under the control of the aP2 promoter.

FIG. 8 depicts the effects of consuming calcium and selective dairy components for six weeks on Gastrocnemius Mass in transgenic mice expressing agouti specifically in adipocytes under the control of the aP2 promoter.

FIG. 9 shows the effects of consuming calcium and selective dairy components, including fresh whey products, for six weeks on weight change in transgenic mice expressing agouti specifically in adipocytes under the control of the aP2 promoter.

FIG. 10 illustrates the effects of consuming calcium and selective dairy components including fresh whey products, for six weeks on Pat Pad Mass in transgenic mice expressing agouti specifically in adipocytes under the control of the aP2 promoter.

FIG. 11 depicts the effects of consuming calcium and selective dairy components including fresh whey products, for six weeks on Gastrocnemius Mass in transgenic mice expressing agouti specifically in adipocytes under the control of the aP2 promoter.

### DESCRIPTION OF THE INVENTION

The calcium-containing fresh whey products of the present invention may be whey or a composition containing whey, whey-derived protein product, whey-derived mineral isolate blends, or combinations thereof, any of which may optionally include intrinsic or additional enzyme-inhibiting peptides, such as angiotensin converting enzyme (ACE) inhibiting peptides.

Whey is generally known as the watery part of milk that is separated from the coagulable part or curd especially in the process of making cheese (Merriam-Webster Online Dictionary <www.m-w.com>). Whey is readily available as a by-product from the dairy industry, which typically is prepared by removing fat and casein from milk, and contains lactose, some salts, and a variety of proteins, including bovine serum albumin “BSA” (molecular weight 66,000 Daltons), alpha-lactoglobulin (molecular weight 14,000 Daltons), beta-lactoglobulin (molecular weight 16,000 Daltons), kappa-casein fragment 106-109, lactoferrin, lactoperoxidase, and immunoglobulins. Salts contained in whey or milk may include calcium, copper, magnesium, phosphorus, potassium, selenium and/or zinc. Whey protein preparations are commercially available, for example, as whey protein concentrates (WPC), which comprises protein and carbohydrate (lactose), e.g., AVONLAC 134 (Glanbia Nutritionals, Inc. of Monroe, Wis. (“Glanbia”)) or whey protein isolates (WPI), e.g., PROVON 190 or 290 (Glanbia) or PROTARMOR (Armor Proteines SAS, France). Other whey providers are Davisco (Le Sueur, Minn.); Bio-Isolates PLC (Deeside, UK); NZMP North America (Santa Rosa Calif.); Formost Farms (Baraboo Wis.); Arla Foods (Union N.J.); Avenmore Waterford (Monroe Wis.); Borculo Fresh whey products; and Borculo, Netherlands.

A fresh whey product according to the invention may be any such fraction derived from any dairy product such as milk or cheese whey. The fresh whey product may be any product produced from whey after fractionation or isolation from milk, including whey, WPC, WPI, whey mineral concentrate, such as TRUCAL calcium isolate (Glanbia) or VITALARMOR (Armor Proteines SAS), containing 24% calcium and 13% phosphorus. The fresh whey product may be obtained by any method known in the art. For example, whey fractions may be obtained by one or more of extraction, ultrafiltration, electrodialysis, evaporation, and reverse osmosis of milk or cheese whey.

The fresh whey product may be in a composition that includes enzyme-inhibiting peptides, which may be derived from such peptides as casein, whey proteins, soy proteins, or any other suitable source via processing means known to those of skill in the art. One example of such peptide is one that inhibits angiotensin converting enzyme (ACE). For example, TRUCAL FP (Glanbia) or BIPRO (Davisco) contains dairy mineral and ACE inhibiting peptide.

The fresh whey product may be administered to an individual in any form known in the art to carry out the purpose of the invention.

Whey products may be damaged at various stages, e.g., manufacture, storage, or shipping. Such products are not considered to be “fresh whey.” A whey product may be stable and maintained as fresh e.g., by not mishandling the raw or finished product, maintaining an appropriate pH, using suitable combinations of aqueous liquids and solid concentrations (e.g., dry whey protein concentrate, liquid whey protein concentrate, whey protein isolate, liquid or dried sweet whey, liquid or dried acid whey), preventing the action of putrefying microorganisms with suitable storage conditions or preservatives, refining or subjecting to, e.g., ultrafiltration, demineralization as by electrodialysis, and blending with other whey derived ingredients, stabilizing with specific components, stabilizing after the fresh whey product is made, e.g., by packaging, storing or maintaining in specific environments, packaging, or temperatures, or functionally enhancing the whey product.

As used herein, “fresh” whey may have substantially undenatured whey protein content, and may be undegraded and undenatured. It may be bioavailable and digestable, meaning that it is not coagulated, clumped, crystallized, or in another form that is undigestable or unavailable biologically or chemically. It has not been heat damaged and is uncontaminated; has not been subject to the putrefying action of putrefying microorganisms at various temperatures, e.g., 5, 23, or 37 degrees Celsius; has not been deemed damaged or rendered ineffective due to exposure to elevated levels of heat, moisture, or exposure to microbial decay. Fresh whey products are fresh as consumed, and thus are stable under the conditions used for manufacture, storage, shipment, and use. A fresh whey product may be stabilized to resist loss of freshness under such conditions, to enhance its stability. Various known compositions of fresh whey or methods of stabilizing whey may be used, e.g., as set forth in U.S. Pat. No. 4,081,555 (Sawhill), U.S. Pat. No. 4,497,836 (Marquardt, et al.), U.S. Pat. No. 4,748,034 (de Rham), U.S. Pat. No. 6,558,716 (Kent, et al.), or U.S. Published Patent Application No. 2003009976 (Jost), which are all incorporated herein by reference.

Fresh whey may also be referred to as stable whey. Without being bound by any theories, fresh whey products may be advantageous because whey products that are not fresh lack the components that help maintain lean body mass as compared to other sources of dietary calcium such as milk and the casein fraction of milk (which predominates in cheeses). Such desirable characteristics of fresh whey may include whey's leucine or branched amino acid content, which is higher than the casein fraction of milk.

In an exemplary embodiment, the fresh whey product is a composition containing whey-derived protein isolate with about 25% to about 99% protein, or at least about 75%, 80%, 85%, 90%, 95%, or greater of protein. The composition may additionally contain less than about 10%, 5%, or 1% of carbohydrates, lactose, fat or other minerals. In another aspect, the protein isolate may contain stabilizers or emulsifiers such as lecithin, ethoxylated mono and diglycerides, and propylene glycol.

In an exemplary embodiment, the fresh whey product is a composition containing whey-derived mineral fraction comprising from about 1-99% whey derived mineral comprising from about 1-30%, 10-90%, 20-80%, 30-70%, 15-30%, 20-30%, 22-28%, or 23-25% calcium.

In another exemplary embodiment, the fresh whey product is a composition containing whey-derived mineral fraction and whey-derived protein isolate, e.g., in the above-stated amounts, for example at least about 78% protein, at least about 15% total minerals and at least about 3.8% calcium.

The fresh whey product may fortify calcium-containing products. That is, the fresh whey product is added to and becomes part of another product. Examples of calcium-containing products that may be fortified with fresh whey products include dietary calcium, calcium carbonate preparations, and other products derived from dairy, such as milk, cream, yogurt or cheese. The calcium-containing product may be, e.g., yogurt or a product derived from yogurt, cheese or a product derived from cheese, or milk or a product derived from milk, such as skim milk, 1% milk, 2% milk, whole milk, half and half or whipping cream. In another exemplary embodiment, the fresh whey product is in the form of a powder or is incorporated into a nutritional or dietary composition or supplement or calcium fortified vitamin supplements, or is incorporated into a food product or foodstuff or other foods high in calcium or any food product that is consumed by the individual. The foodstuff may be based on dairy, eggs, grains, legumes, vegetables, fruits, meat, fish, or poultry. It may be, for example, cereal, salmon, beans, tofu, spinach, turnip greens, kale, broccoli, waffles, pancakes, pizza, cottage cheese, ice cream or frozen yogurt. In another exemplary embodiment, the calcium-containing product comprises infant formula, nutriceuticals, or meal replacement beverages or drinks or powders for preparing any of the above.

In another embodiment, the product may be in the form of a pill, tablet, capsule, or combination with other minerals and/or vitamins. The product may be for human or non-human/animal consumption, such as pet food or farm/agricultural animal feed.

The present invention also provides methods of increasing the amount of fresh whey product ingested or consumed by the individual and, optionally, in combination with other dietary efforts, e.g., restricting the caloric intake or exercising, and/or administering the fresh whey product for a prolonged period of time to obtain the desired results of inducing weight and/or fat loss, preventing weight and/or fat gain, and/or increasing the metabolic consumption of adipose tissue. For example, the product may be administered for a continuous interval of at least about one week, two weeks, three weeks, one month, six weeks, two months, three months, six months, or one year, wherein the product is administered on an average daily basis in amounts effective to induce the desired results.

The amount effective to induce the desired results may be based on the amount of calcium contained in said fresh whey product, e.g., at least about 1000 mg calcium per day. A fresh whey product or a serving thereof may contain on average at least about 100 mg, 200 mg, 255 mg, 300 mg, 400 mg or 500 mg. Fresh whey product consumption may provide calcium consumption of at least about 500 mg, 600 mg, 773 mg, 800 mg, 900, mg, 1000 mg, 1100 mg, 1200 mg, 1300 mg, 1346 mg, 1400 mg, 1500 mg, 1600 mg, or higher amounts, on average, for at least two weeks, one, two, three months, or longer. In an exemplary embodiment, the fresh whey product is administered over a continuous interval for at least one month, providing at least about 1000 mg of calcium on an average daily basis. These dosages may be referred as being “high calcium.”

Another aspect of the invention provides for methods of determining dietary calcium consumption of the individual, wherein the individual is a human and (1) if the dietary calcium consumption is below 1000 mg/day, increasing the fresh whey product consumption, and (2) if the dietary calcium consumption is at least about 1000 mg/day, maintaining that level through fresh whey product consumption. In another embodiment the amount of calcium consumed by the individual before administering the effective amount of fresh whey product is less than about 400 mg/day, 600 mg/day or 773 mg/day of calcium. In another aspect, the average daily fresh whey product administered may provide at least about, 800 mg/day, 1000 mg/day, 1100 mg/day, 1200 mg/day, 1346 mg/day, or 1400 mg/day of calcium.

In yet another exemplary embodiment, the effective amount of calcium-containing fresh whey product may be in the form of a dairy product fortified with the fresh whey product which is administered in at least about 3 to 4 servings per day, e.g., 2.5 or 3.5 servings. An example of serving size may comprise whey fortified product having the calcium content of at least about 8 ounces of milk, 8 ounces of yogurt or 1.5 ounces of cheese. In an exemplary embodiment, a serving may contain at least about 200, 255 or 300 mg, two servings may contain about 700, 773 or 800 mg, and three servings may contain 1000, 1100, 1300, 1346 or 1400 mg of calcium. In one aspect, the serving portion may contain on average at least about 300 mg of whey-derived dietary calcium. In another aspect of the invention, a serving portion may contain on average at least about 200 mg of dietary calcium. In an exemplary embodiment, the effective amount of whey-derived dietary calcium is at least about 1000 mg per day. In another embodiment, the fresh whey product may be administered on an average daily basis equaling to at least about 50 to 75 servings per month or 100 to 120 servings per month. In an embodiment, the amount of calcium is administered on average at about 1346 mg a day, or 3 to 3.5 servings of fresh whey product to achieve the desired effects of inducing weight and/or fat loss, preventing weight and/or fat gain, and/or increasing the metabolic consumption of adipose tissue.

The fresh whey product of the present invention provides high calcium bioavailability and is more effective than mineral calcium (e.g. from calcium carbonate) for regulating body weight and/or body fat by inducing weight and/or fat loss, preventing weight and/or fat gain, and/or increasing the metabolic consumption of adipose tissue in the individual. The fresh whey is more effective than non-fresh whey products (non-stable whey products). It is almost or at least as effective as milk and more effective than the casein fraction of milk at managing body mass.

The methods and compositions of the invention are effective at managing body mass, e.g. regulating or controlling body weight, adiposity, and muscle mass. They are also effective in preventing, reducing the severity of, reducing the risk of, or treating a body weight disorder or other disorder that is associated with being overweight such as obesity, high blood pressure, stroke, premenstrual syndrome, depression, hypertensive disorders of pregnancy, Type-2 diabetes, asthma, inflammatory bowel disease, attention deficit disorder, migraine headaches, kidney disease, kidney stones, hypercholesterolemia, congestive heart failure, immune deficiency, and others known to practitioners.

Example 1, which uses some non-fresh whey products, is included for comparison purposes to Example 2, which uses a fresh whey product. Example 1 is the subject of co-pending U.S. provisional application, No. 60/558,706, owned by the same entity as this application.

The inventor previously discovered that calcium-containing products, such as milk, are effective for inducing weight and/or fat loss. As set forth and detailed in Examples 1 and 2, surprisingly, calcium-containing whey products and fresh whey products are more effective than calcium in the same amounts from mineral sources, such as calcium carbonate, at inducing weight loss, preventing weight and/or increasing the metabolic consumption of adipose tissue in an animal. Diets with fresh whey, whey protein, whey derived mineral isolate or blends, or whey derived mineral isolate with enzyme-inhibiting peptides, can be more effective than administering calcium alone or calcium with other non-whey or non-fresh whey containing products in inducing weight and/or fat loss.

The present invention may also include maintaining the individual on a caloric restricted or energy restricted diets. Often such restricted diets results in both fat and muscle loss. The present invention provides for a method of weight regulation by selectively targeting specific regions of the body and/or types of fat or muscle cells or tissue.

In an exemplary embodiment, the present invention is directed to methods of providing weight related diets, e.g., that selectively target specific fat cell or tissue zones while maintaining lean or muscle mass, by administering fresh whey products to an individual and thereby augmenting weight loss, while protecting muscle or lean body mass. The fresh whey products may also help the body recover rapidly from the metabolic stress of exercise and help prevent individuals from gaining extra pounds of fat. These whey-based diets promote a repartitioning of dietary energy from adipose tissue to skeletal muscle, resulting in preservation and/or improvement in lean body mass while adipose tissue is lost.

Fresh whey products administered according to the invention promote apoptosis in adipocytes. That is, a diet comprising fresh whey products as part of a sufficient level of calcium induces programmed cell death in fat cells, and can reduce the number of fat cells in the dieting individual. The reduction can be preferentially in the abdominal or truncal region, and contributes to loss of fat mass. The reduction in fat cell number may be in the range of at least about 5%, 10%, 20%, or 30% of adipocytes. Thus, a fresh whey product diet according to the invention can be promoted as killing, eliminating, or reducing the number of fat cells.

For example, one embodiment provides a method comprising administering to an individual regulating body weight a calcium containing fresh whey product in an amount effective to maintain muscle mass while inducing weight and/or fat loss, preventing weight and/or fat gain, and/or increasing the metabolic consumption of adipose tissue in the individual.

The retention of lean or muscle mass may be maintained selectively, in various regions of the body, e.g., in skeletal muscle, in the limbs (such as arms and legs) or the trunk. For example, the consumption of fresh whey products can maintain gastrocnemius muscle. Likewise, inducement of fat loss may also be selectively targeted in various regions or types, e.g., loss of fat pad mass, abdominal-fat, perirenal fat or subscapular fat and/or combinations. The present invention may also assist in inducing fat loss while maintaining lean or muscle mass. Maintaining muscle mass may include gaining at least about 1, 2, 5, or 10% muscle mass, or not losing any muscle mass, or losing no more than about 1%, 2%, or 5% of muscle mass during the dieting period during which fresh whey products are consumed.

The efficacy of diets containing various constituents for limiting weight and fat was measured to be approximately as follows, from least to most effective constituents: low calcium, high mineral calcium, whey mineral isolate, and whey protein and milk. ACE inhibitors improved the efficacy of whey mineral isolate and whey protein diets.

Example 2 shows that fresh whey products, e.g., in Group 4, are as effective as milk products, e.g., Group 3, and that fresh whey products containing whey-derived mineral fractions are at least as effective at regulating adiposity as other supplementary calcium sources (e.g. calcium carbonate).

The data also show that whey surprisingly retains most or all of the bioactivity found in milk and that the whey-derived ACE inhibitor contributes to this effect. The ACE inhibitory action causes a consistent improvement in adiposity, weight loss, etc., but intact fresh whey has a much larger effect.

In another exemplary embodiment, the invention is directed to compositions and methods for maintaining a predetermined body weight range and treating an overweight condition or obesity by enhancing weight loss and/or limiting weight gain and promoting good health. Overweight and obesity has been associated to some degree with inadequate intake of dairy products, and more particularly the minerals present in dairy products. It has been discovered that an overweight condition and obesity can be effectively treated by administering nutritional supplement compositions, either directly or via food products fortified with the compositions, in accordance with the practice of the present invention. The nutritional supplement compositions contain therapeutically effective amounts of whey mineral, whey protein and/or whey-derived enzyme-inhibiting peptides and are administered prior to or during a meal. The nutritional supplement compositions also can be administered to an individual seeking to maintain a desired body weight.

Treatment can be enhanced by use of additional ingredients in the composition to address other mechanisms for weight control. Additionally, enzyme-inhibiting peptides may be included to assist with regulation of adiposity by controlling fat metabolism.

The terms “treat,” “treating,” “treatment,” and similar terms as used herein refer to the administration of the nutritional supplement compositions to individuals, particularly humans, who are overweight or obese, for alleviating, suppressing, inhibiting, or otherwise reducing the extent to which the individual is overweight or obese or any symptom associated therewith. The terms “treat,” “treating,” “treatment,” and similar terms also are used herein to refer to the prophylactic administration of the nutritional supplement compositions to individuals who may be at risk of, or otherwise wish to avoid, becoming overweight or obese.

One component of the nutritional supplement composition is whey minerals. See U.S. Published Patent Application No. 2003/0165574, published Sep. 4, 2003, Ward et al., incorporated herein by reference. Commercially available milk mineral products may include, e.g., TRUCAL products (Glanbia). An example of a composition of whey mineral is illustrated in Table 1 below.

TABLE 1
Composition for Whey Mineral Powder

| Component | Relative Amount (% by weight) |
|---|---|
| Total Minerals | 50-90% |
| Inorganic Mineral (Ash) | 45-85% |
| Organic Mineral (Citrate) | 1-10% |
| Calcium | 15-35% |
| Magnesium | 0-10% |
| Phosphorous | 7-15% |
| Potassium | 0-5% |
| Zinc | 0-1% |
| Lactose | 0-15% |
| Protein | 1-15% |
| Free Moisture | 2-5% |
| Fat | 0-5% |

In another aspect of the present invention, the fresh whey composition includes whey proteins. Whey proteins occur in milk as soluble, globular proteins and are different than the suspended casein particles. Generally, they are an important source of protein needed for overall good health and nutrition. The primary proteins and peptide constituents derived from whey proteins include alpha-lactalbumin and beta-lactoglobulin, kappa-casein, lactoferrin, bovine serum albumin, lactoperoxidase, and immunoglobulins.

In another aspect of the present invention, the fresh whey composition includes active enzyme-inhibiting peptides. Examples include peptides that inhibit angiotensin converting enzyme (ACE). Angiotensin II is a hormone that is synthesized and secreted by adipose cells. Literature has shown that angiotensin II may be involved in control of adiposity through regulation of lipid synthesis and storage of adipocytes. Some dairy peptides are associated with the inhibition of angiotensin converting enzyme (ACE). Thus, by administering fresh whey containing a therapeutically effective amount of ACE-inhibiting peptides, weight loss can be enhanced. Without intending to be limited by theory, it is possible that non-fresh whey products have lower levels of enzyme-inhibiting peptides such as ACE inhibitors, as compared to fresh whey. To obtain the compositions of the present invention, the components selected for the compositions can be processed as desired prior to preparation of the nutritional supplement compositions, in a manner that maintains the stability or freshness of the whey product. Whey mineral extract typically is purified, spray dried, and ground into a powder having an appropriate particle size to permit mixing with a liquid or solid food product if desired. Similarly, the protein component typically is purified, dried, and ground into a powder. ACE-inhibiting peptides also can be provided in the composition. A dietary supplement or food composition according to the invention may optionally include other ingredients, such as minerals, vitamins, flavorings and colorants, in accordance with techniques well known to persons skilled in the art.

Suitable particle sizes for the composition will depend on such factors as the physical properties (e.g., liquid or solid, specific gravity, pH, viscosity, etc.) of the food product into which the powder is mixed, and the need to maintain a stable fresh whey product.

Fresh whey products can be used according to the invention as an additive for a wide variety of types of food products, and food product compositions containing fresh whey products according to the invention include acidic juice and sport beverages, neutral pH beverages (e.g., milk UHT dairy, RTD nutritional, soy milk, or shakes and other blended beverages such as milkshakes, smoothies, frappes), nutritional supplement foodstuffs (e.g., high-energy protein bars), confectionery products (e.g., high calcium chews, chewing gum, chocolate, or cookies), dairy products (e.g., yogurt, ice cream, milk, cheese, processed cheese, or butter), and farinaceous products (e.g., bread, muffins, biscuits, cereal or rolls). The relative amount by weight of the fresh whey compositions combined with a food product depends on such factors as the density and the serving size of the food product. Typically, the amount of fresh whey compositions may range from 0.1 to about 10 percent by weight based on the total weight of the food product.

The formulation of the composition and, if administered via a food product, the amount of the composition blended into the food product, are selected to provide desired amounts of the particular components so as to be effective for managing body mass. By way of example, a typical nutritional supplement composition may be administered to provide between at least about 1, 2, 5, 10, or 15 grams of protein, at least about 0.2, about 0.5 or about 6 grams or more of calcium, and at least about 0.1, 0.5, 1, 2, 3, 5, or 10 grams or more of ACE-inhibiting peptides per serving of the composition. The amount of composition administered can be adjusted as desired to account for differences in physical characteristics and nutritional requirements and body mass index of the individuals to whom the composition is administered.

In accordance with the methods of the present invention, body weight conditions including overweight and obesity are effectively managed and treated. That is, an individual of healthy condition and having a generally ideal weight can manage his or her weight and maintain a desired weight range. An individual who has a weight in excess of a desired range and may be considered overweight or obese can be effectively treated by limiting weight gain and/or promoting weight loss while maintaining muscle mass. A therapeutically effective amount of the nutritional supplement composition is administered to an individual to provide these benefits.

The present invention may comprise methods of administering fresh whey products to induce weight loss, maintain body weight or increase muscle retention. In an exemplary embodiment, the caloric intake of an individual may be unmodified or ad lib or it may be desirable to reduce the caloric intake of the individual as part of a dietary plan. The restriction of unmodified or ad lib caloric intake may be slight or more extensive based on whether the individual is maintaining or reducing body weight. For example, an individual may maintain body weight and/or increase muscle retention and increase fat loss, by consuming fresh whey products with unmodified caloric intake, e.g., ad lib (unrestricted intake of food or calories available to the point of satisfaction). However, an individual may increase fat or weight loss by restricting caloric intake below ad lib. Examples of caloric restriction may be reducing caloric intake by about 200 to 500 calories/day, e.g., 100, 200, 300, 400, 500, 600, 700, 800, 900, 1000, 1200 or 1500 calories/day as opposed to unmodified or ad lib. The individual may also restrict the amount of caloric intake to a total of about 500 to about 5000 calories/day, or less then about 4500, 4000, 3500, 3000, 2500, 2000, 1500, or 1000 calories/per day.

In another exemplary embodiment, the invention may relate to a weight management composition comprising therapeutically effective amounts of fresh whey product containing components selected from the group consisting of whey-derived mineral fraction, whey-derived protein isolate and whey-derived enzyme-inhibiting peptide fraction, in a package labeled with information or a description about the effects of consuming effective amounts of the whey product to maintain and/or increase muscle mass while inducing weight and/or fat loss, preventing weight and/or fat gain, and/or increasing the metabolic consumption of adipose tissue in the individual. In one aspect of the invention, the information or description is in the form of printed matter or material. In a further aspect, the product is packaged and the description is part of the package, directly accompanies the product as an insert or label, or is imprinted on the product. The labeling may also be in the form of notices or information associated with the product package, for example on store shelves, in store aisles, or in notices or advertisements in any standard print or electronic media, including the internet, television, or radio.

In another exemplary embodiment, the invention relates to a method comprising communicating to a potential consumer that consuming fresh whey products may have a beneficial effect on body weight or body fat and/or muscle retention, the communicating being by an entity having a commercial interest in the consumption of the product. In another aspect, the invention may relate to a method for inducing the consumption of fresh whey products by a commercial entity having a financial interest in the sale of the products, wherein the entity distributes information to potential consumers of the fresh whey products describing weight-control and/or muscle retention benefits of fresh whey products attributable to the consumption of the products. In yet a further aspect, the invention may be a method for promoting the consumption of a fresh whey product wherein said method comprises the public distribution of information describing the weight or obesity-control benefits of said product which are attributable to the consumption of calcium in said product.

In one aspect of the invention, the communicating, the distribution of information or the public distribution may be by verbal communication, pamphlet distribution, print media, audio tapes, magnetic media, digital media, audiovisual media, billboards, advertising, newspapers, magazines, direct mailings, radio, television, internet websites, electronic mail, electronic media, banner ads, fiber optics, and information on a product package, insert or label or directly accompanying the product.

In another exemplary embodiment, the invention may be a method of regulating the body weight of an individual comprising the steps of: (a) providing the individual with information disclosing that consuming fresh whey products is associated with one or more health effects selected from loss and/or reduced gain of weight and/or fat, and/or metabolic consumption of adipose tissue and/or muscle retention, and (b) providing the individual with a dietary plan for consuming products containing an effective amount of fresh whey products sufficient to induce the one or more health effects.

In another exemplary embodiment, the invention may be a method of avoiding or reducing the risk of health problems in an individual at risk thereof due to excess body weight and/or an excess of body fat, comprising administering to the individual a sufficient amount of fresh whey products to induce a metabolic change as compared to suboptimal amounts of fresh whey consumption. In one aspect, the health problem may be one or more of coronary artery disease, stroke, osteoporosis, osteoarthritis, ligament injuries, perineal dermatitis, diabetes mellitus, cardiomyopathy, and/or urologic syndrome.

In another exemplary embodiment, the invention relates to a method of regulating weight comprising administering fresh whey products in an amount sufficient to induce weight loss, prevent weight gain, and/or increase the metabolic consumption of adipose tissue and/or muscle retention in a non-human animal. In one aspect, the animal may be a mammalian pet, e.g., dog or cat, or a mouse or farm animal. In another aspect, the invention may be an animal food package comprising a fresh whey product animal food and a description of the benefits of consuming fresh whey in inducing weight loss, preventing weight gain, and/or increasing the metabolic consumption of adipose tissue and/or muscle retention. The animal food may be pet food or farm animal feed.

### EXAMPLES

### Example 1

This example relates to the fraction(s) of dairy responsible for dairy-induced augmentation of the anti-obesity muscle mass retaining effect of calcium in diet-induced obese transgenic mice. Specifically, it relates to the effectiveness of whey-based high calcium diets versus casein-, soy- and milk-based high calcium diets (compared to a low calcium control) in accelerating weight and fat loss secondary to caloric restriction in agouti-transgenic mice fed high fat/high sucrose/low fat diets, and to whether the augmented anti-obesity effect of whey is attributable to whey protein(s) or the mixture of minerals, in addition to calcium, found in whey.

### Animal and Diets

To evaluate the role of dietary calcium in regulating adiposity in vivo, transgenic mice expressing agouti specifically in adipocytes under the control of the aP2 promoter were studied. These animals exhibit a normal pattern of leptin expression and activity similar to that found in humans and exhibit a human pattern (adipocyte-specific) of agouti expression. These mice are useful models for diet-induced obesity in that they are not obese on a standard AIN-93G diet, but become obese in response to hyperinsulinemia induced by either insulin administration or high sucrose diets. In separate studies it has been confirmed that this animal model is predictive for nutritional effects in humans regarding body mass, including weight loss, weight gain, fat mass, and muscle mass.

### Methods

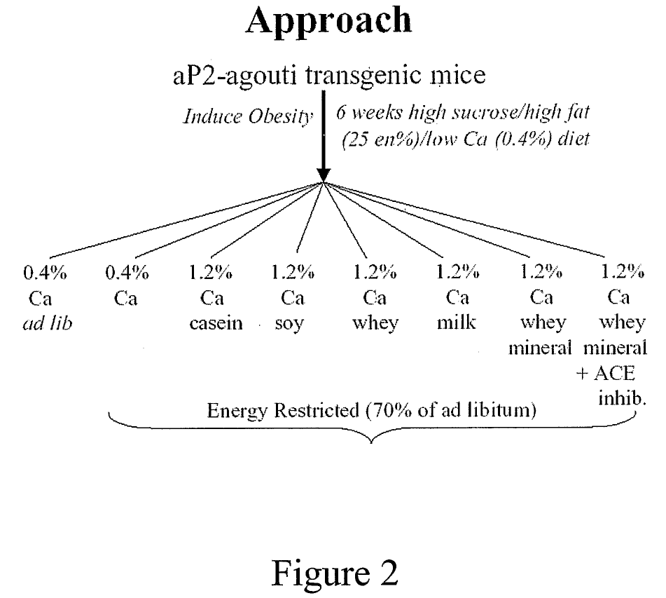

As illustrated in FIG. 2, mice were fed a low calcium (0.4%), high sucrose, high fat diet for 6 weeks to induce obesity. All diets had 25% fat (lard+soy oil), with sucrose as sole carbohydrate and the proteins indicated as the sole protein source. Group 1 continued for 6 weeks on the above diet ad libitum.

Groups 2-8 continued for six weeks on a restricted (70% of the ad libitum energy intake). Group 2 was maintained at a low calcium diet (casein/0.4% calcium) comprising protein from milk casein and 0.4% calcium from CaCO₃.

Groups 3-6 continued on high calcium, energy restricted diets. Group 3 was maintained on non-fat dry milk/1.2% calcium, comprising dried milk having 1.2% calcium, most of which comes from milk, with some from CaCO₃. Group 4 was maintained on whey protein isolate/1.2% calcium, comprising only protein from whey and calcium mostly from whey with a modest amount from CaCO₃ (e.g., PROVON 190). Group 5 was maintained on casein/1.2% calcium, comprising protein from casein and 1.2% calcium from CaCO₃. Group 6 was maintained on soy protein isolate/1.2% calcium, comprising protein from soy and 1.2% calcium from CaCO₃.

Groups 7 and 8 continued on high calcium (1.2%), energy restricted diets. Group 7 used a whey mineral isolate, containing 1.2% calcium and protein from casein without a whey-derived angiotensin converting enzyme (ACE)-inhibitor (TRUE-CAL). Group 8 used a whey mineral isolate with a whey-derived ACE-inhibitor (TRUE-CAL FP).

During the six weeks, body weight, food intake and core temperature were measured weekly. At the end of six weeks, fat pad mass, adipocyte intracellular Ca²⁺([Ca²⁺]ᵢ), fatty acid synthase expression and activity and lipolysis was assessed.

### Results

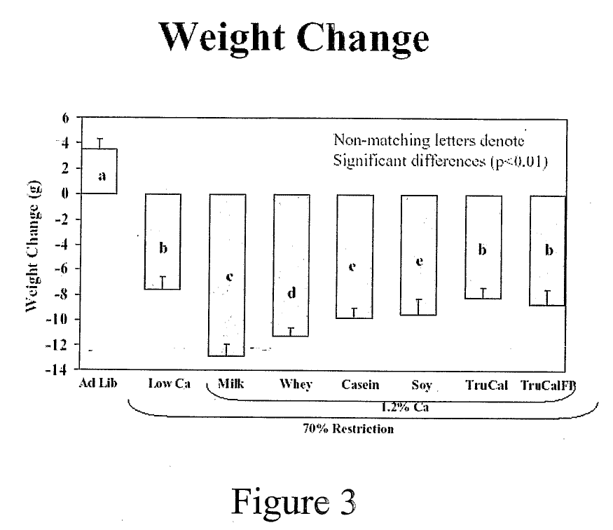

FIG. 3 demonstrates that high calcium milk Group 3 mice had the most significant and substantial weight change, followed by high calcium whey protein isolate Group 4. High calcium casein Group 5 and soy Group 6 had less weight change than milk or whey protein isolate but more than low calcium Group 2 and high calcium whey mineral Group 7 and whey mineral/ACE Group 8. The least amount of weight change was in the Ad Lib Group 1.

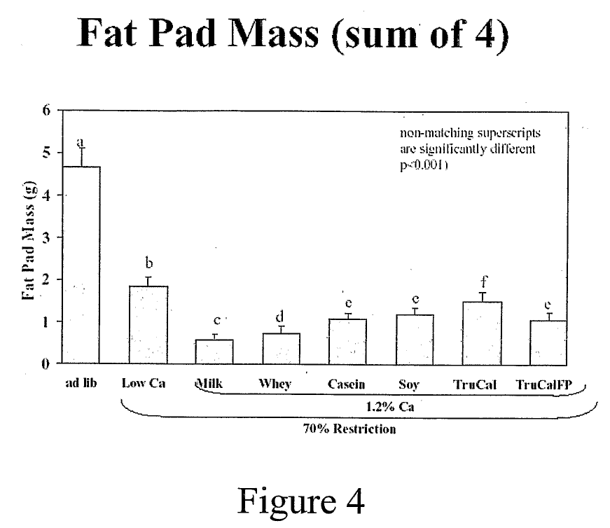

As observed in FIG. 4, Ad Lib Group 1 mice had significantly more fat pad mass than any of the other calcium containing groups. The least fat pad mass was seen in high calcium milk Group 3, followed by high calcium whey protein isolate Group 4. High calcium casein Group 5, soy Group 6 and whey mineral/ACE Group 8 had more fat pad mass than milk or whey protein isolate but less than high calcium whey mineral Group 7, which had even less than low calcium Group 2.

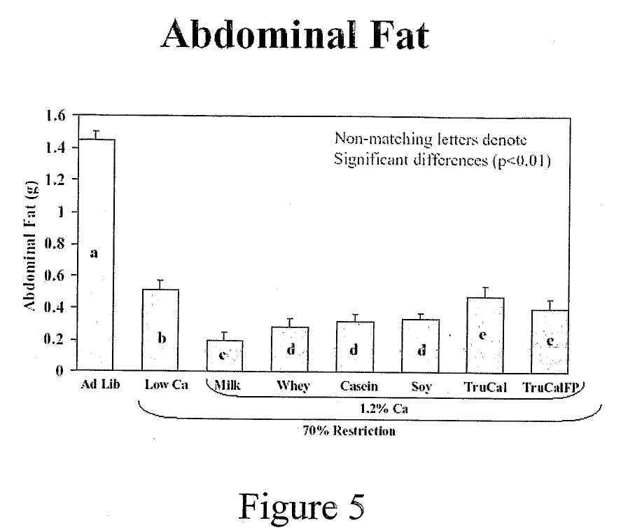

In FIG. 5, Ad Lib Group 1 mice appear to have significantly more abdominal fat than any of the other calcium containing groups. The least abdominal fat was observed in high calcium milk Group 3, followed by high calcium whey protein isolate Group 4, high calcium casein Group 5 and soy Group 6. High calcium whey mineral Group 7 and whey mineral/ACE Group 8 had more abdominal fat than the other high calcium containing groups, but less than low calcium Group 2.

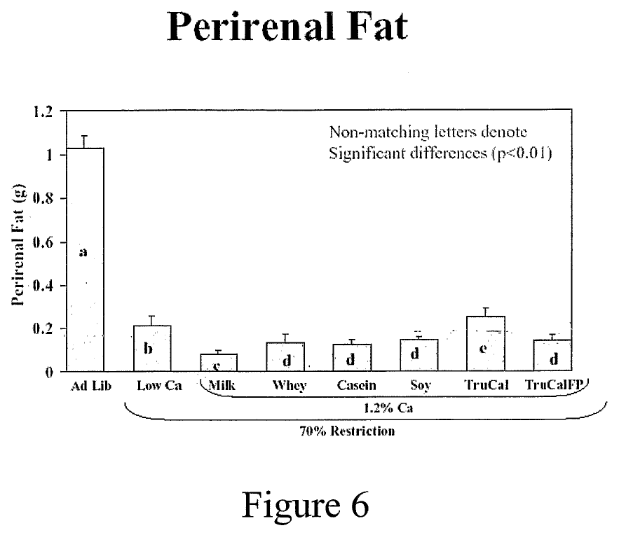

FIG. 6 shows that Ad Lib Group 1 mice appear to have significantly more perirenal fat than any of the other calcium containing groups. The least perirenal fat was observed in high calcium milk Group 3, followed by high calcium whey protein isolate Group 4, high calcium casein Group 5, soy Group 6 and whey mineral/ACE Group 8. Low calcium Group 2 had more perirenal fat than the other high calcium containing groups, but less than high calcium whey mineral Group 7.

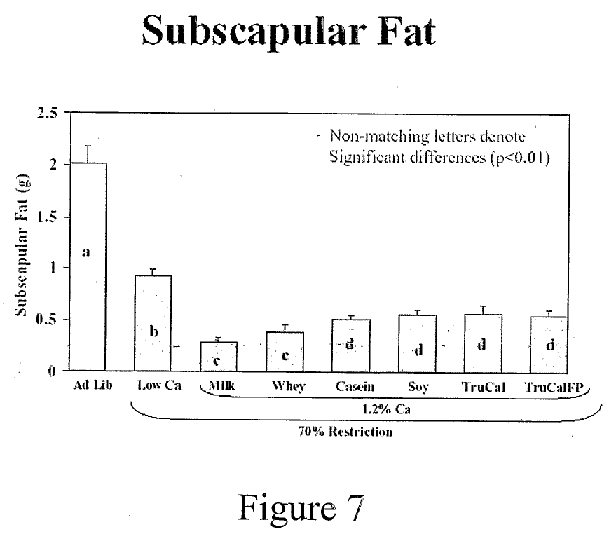

As seen in FIG. 7, Ad Lib Group 1 mice had significantly more subscapular fat than any of the other calcium containing groups. The least subscapular fat was seen in high calcium milk Group 3 and whey protein isolate Group 4, followed by high calcium casein Group 5, soy Group 6, whey mineral Group 7 and whey mineral/ACE Group 8, which had even less than low calcium Group 2.

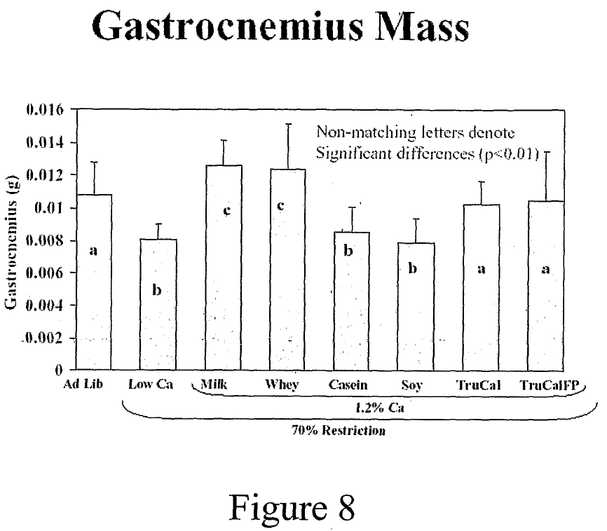

In FIG. 8, high calcium milk Group 3 and whey protein isolate Group 4 had the most gastrocnemius muscle mass, followed by high calcium casein Group 5, soy Group 6 and low calcium Group 2. High calcium whey mineral Group 7 and whey mineral/ACE Group 8 had less than all the other groups, except for Ad Lib Group 1.

Table 2 summarizes these results.

TABLE 2

| Weight Management Indication | Benefit Ranking |
|---|---|
| weight change (most to least) | 3 > 4 > 5 = 6 > 2 = 7 = 8 > 1 |
| fat pad mass (least to most) | 3 > 4 > 5 = 6 = 8 > 7 > 2 > 1 |
| abdominal fat (least to most) | 3 > 4 = 5 = 6 > 7 = 8 > 2 > 1 |
| perirenal fat (least to most) | 3 > 4 = 5 = 6 = 8 > 2 > 7 > 1 |
| subscapular fat (least to most) | 3 > 4 = 5 = 6 = 7 = 8 > 2 > 1 |
| gastrocnemius muscle mass (most to least) | 3 = 4 > 5 = 6 = 2 > 7 = 8 > 1 |

1 = Ad Lib
2 = loCa
3 = milk
4 = whey protein
5 = casein
6 = soy
7 = whey mineral
8 = whey mineral/ACE

### CONCLUSION

As demonstrated here, high calcium diets caused a 32% augmentation of fat loss versus the low calcium diet (p<0.0001), while milk augmented this fat loss by 62.5% vs. the other high calcium, diets (p<0.001). Whey was not as effective as milk, and the whey minerals used provided no additional fat loss. Addition of the whey ACE inhibitor reduced fat mass by 22% (p<0.001), although intact milk was still markedly more effective, which may be due to an artifact of processing, resulting in compromised bioavailability. Thus, whey components provide an anti-obesity effect.

In addition, energy restriction on the low calcium diets resulted in a decrease in gastrocnemius muscle mass which was prevented by the whey diet (which produced a modest, significant increase), but not by the calcium-supplemented casein and soy diets.

Whey-based diets appear to promote a repartitioning of dietary energy from adipose tissue to skeletal muscle, resulting in preservation and/or improvement in lean body mass while adipose tissue is lost. Fresh whey products appear to produce the full anti-obesity effect and produced the skeletal muscle sparing effect.

The example shows that inclusion of increased calcium or calcium-rich fresh whey products into the diet of these mice significantly attenuated diet-induced obesity and markedly accelerated both weight and fat loss secondary to caloric restriction. Although these effects were attributable, in part, to calcium, whey-derived sources of calcium exerted greater effect than equivalent amounts of calcium from supplements.

### Example 2

This example compared fresh whey fraction responsible for dairy-induced augmentation of the anti-obesity muscle mass retaining effect of calcium in diet-induced obese transgenic mice. The effectiveness of fresh whey product-based high calcium diets was compared to casein-, soy- and milk-based high calcium diets (compared to a low calcium control) in accelerating weight and fat loss secondary to caloric restriction in agouti-transgenic mice fed high fat/high sucrose/low fat diets.

### Animal and Diets

The same animals were used as in Example 1.

### Methods

As illustrated in FIG. 2, mice were fed a low calcium (0.4%), high sucrose, high fat diet for 6 weeks to induce obesity. All diets had 25% fat (lard+soy oil), with sucrose as sole carbohydrate and the proteins indicated as the sole protein source. Group 1 continued for 6 weeks on the above diet ad libitum.

Groups 2-8 continued for six weeks on a restricted diet (70% of the ad libitum energy intake). Group 2 was maintained at a low calcium diet (casein/0.4% calcium) comprising protein from milk casein and 0.4% calcium from CaCO₃.

Groups 3-6 continued on high calcium, energy restricted diets. Group 3 was maintained on non-fat dry milk/1.2% calcium, comprising dried milk having 1.2% calcium, most of which comes from milk, with some from CaCO₃. Group 4 was maintained on fresh whey protein isolate/1.2% calcium, comprising only protein from fresh whey and calcium mostly from whey with a modest amount from CaCO₃ (e.g., PROTARMOR).

Group 5 was maintained on casein/1.2% calcium, comprising protein from casein and 1.2% calcium from CaCO₃. Group 6 was maintained on soy protein isolate/1.2% calcium, comprising protein from soy and 1.2% calcium from CaCO₃.

Groups 7 and 8 continued on high calcium (1.2%), energy restricted diets. Group 7 used a fresh whey mineral isolate, containing 1.2% calcium and protein from casein without a whey-derived angiotensin converting enzyme (ACE)-inhibitor (e.g., VITALARMOR). Group 8 used a fresh whey mineral isolate with a whey-derived ACE-inhibitor (e.g., BIPRO).

During the six weeks, body weight, food intake and core temperature were measured weekly. At the end of six weeks, fat pad mass, adipocyte intracellular Ca²⁺ ([Ca²⁺]ᵢ), fatty acid synthase expression and activity and lipolysis was assessed.

Specifically, core temperature was monitored weekly as an indirect metabolic index using a thermocouple, and fasting insulin (by radioimmunoassay), leptin (by radioimmunoassay) and glucose (by glucose oxidase) levels were assessed at baseline and at weeks 2, and 6. At the conclusion of the study, all mice were sacrificed under isofluorane anaesthesia and individual subcutaneous and visceral fat depots were excised and weighed for comparison of fat accumulation among the groups and assessment of adipocyte size and number via a Coulter Counter method. Aliquots of adipose tissue were then immediately frozen in liquid nitrogen for subsequent study of gene expression. Other adipose tissue aliquots were utilized immediately for the determination of fatty acid synthase activity, basal and forskolin-stimulated lipolysis and intracellular Ca²⁺.

### Results

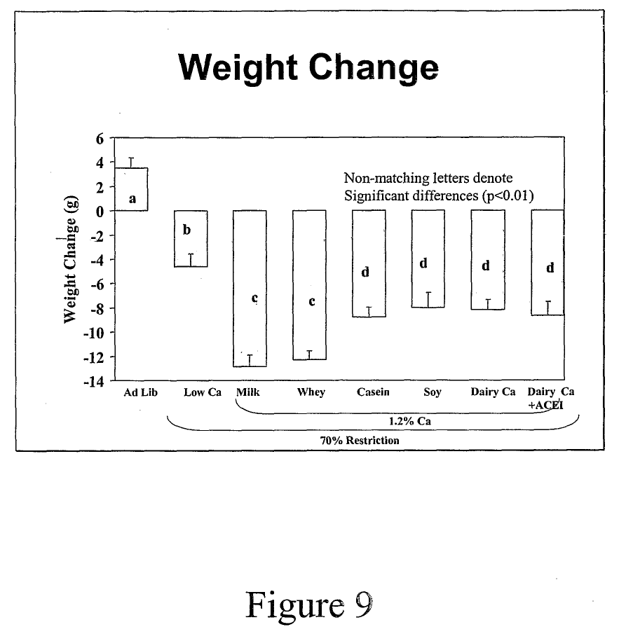

FIG. 9 demonstrates that high calcium milk Group 3 mice and fresh whey protein isolate Group 4 had the most significant and substantial weight change, followed by high calcium. High calcium casein Group 5, soy Group 6, high calcium whey mineral Group 7 and whey mineral/ACE Group 8 had less weight change than milk or whey protein isolate but more than low calcium Group 2. The least amount of weight change was in the Ad Lib Group 1.

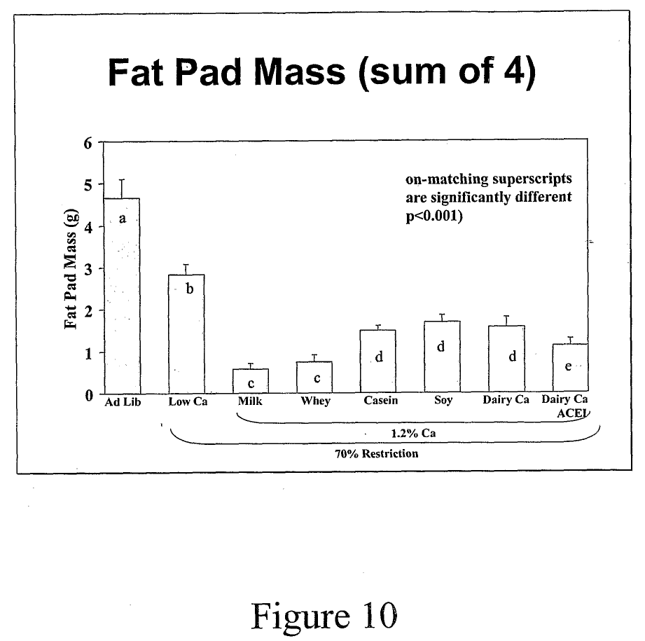

As observed in FIG. 10, Ad Lib Group 1 mice had significantly more fat pad mass than any of the other calcium containing groups. The least fat pad mass was seen in high calcium milk Group 3 and whey protein isolate Group 4, followed by whey mineral/ACE Group 8. High calcium casein Group 5, high calcium soy Group 6, and high calcium whey mineral Group 7 had more fat pad mass than Group 3, 4 or 8, but less than low calcium Group 2.

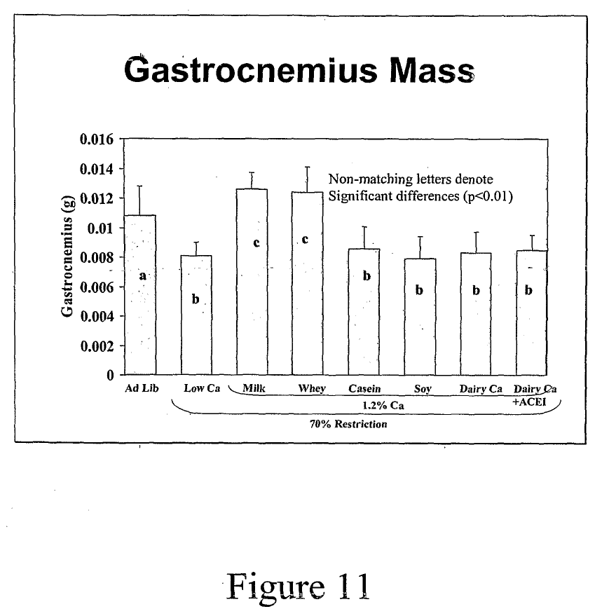

In FIG. 11, high calcium milk Group 3 and fresh whey protein isolate Group 4 had the most gastrocnemius muscle mass, followed by Ad Lib Group 1 (which also had high weight and fat), and then low calcium Group 2, high calcium casein Group 5, soy Group 6, high calcium fresh whey mineral Group 7 and fresh whey mineral/ACE Group 8. Thus, fresh whey protein isolate provided optimal body mass management for lean body mass, balancing low adiposity with high muscle mass.

Table 3 summarizes these results.

TABLE 3

| Weight Management Indication | Benefit Ranking |
|---|---|
| weight change (most to least) | 3 = 4 > 5 = 6 = 7 = 8 > 2 > 1 |
| fat pad mass (least to most) | 3 = 4 > 8 > 5 = 6 = 7 > 2 > 1 |
| gastrocnemius muscle mass (most to least) | 3 = 4 > 1 > 2 = 5 = 6 = 7 = 8 |

1 = Ad Lib
2 = loCa
3 = milk
4 = fresh whey protein
5 = casein
6 = soy
7 = fresh whey mineral
8 = fresh whey mineral/ACE

### Discussion and Conclusion

Overall, these data confirm previous observations of both calcium and dairy accelerating weight and fat loss during calorie restriction, with dairy exerting a markedly greater effect. Surprisingly, fresh whey protein exerted equivalent effects to that of non-fat dry milk, and was superior to non-fresh whey protein as used in Example 1. Fresh whey derived ACE inhibitors were also more effective than in the non-fresh material used in Example 1. Accordingly, the “anti-obesity” factors in milk are found primarily in fresh whey fractions. The animals on the low calcium diets lost significant amounts of muscle mass during calorie restriction, similar to the loss of lean body mass that occurs during dieting in humans. However, the fresh whey-containing diets completely prevented the loss of muscle and actually caused a modest increase in muscle mass despite the caloric restriction, comparable to that achieved with milk. This effect was specific to the fresh whey and milk-based diets, as none of the other high calcium diets exerted such a protective effect on muscle.

The mineral mix derived from whey did not exhibit greater anti-obesity activity than supplemental calcium. Addition of the fresh whey-derived ACE inhibitor to the whey-derived mineral mix significantly increased fat loss, but intact milk and fresh whey were markedly more effective in promoting fat loss. The findings of this study confirm that fresh whey exerts a markedly greater anti-obesity effect than supplementary calcium and demonstrate that virtually all of the “anti-obesity” activity of milk is found in fresh whey fraction. Calcium is clearly partially responsible for this effect, and our data show that a portion of the additional (non-calcium related) activity is attributable to fresh whey-derived ACE inhibitors. Also, the overall effect of dairy is likely to result from a synergistic effect among multiple components of dairy which are found primarily in fresh whey; consequently, it is unlikely that isolated components of dairy are likely to replicate the anti-obesity effect of intact (fresh, stable) whey and/or dairy products. Moreover, the anti-obesity agents present in fresh whey protein and peptide preparations are apparently degraded, absent, or unavailable in some commercially available whey preparations. Accordingly, fresh whey is unexpectedly advantageous as a dietary component for body mass management. Although energy restriction on the low calcium basal diet produced a predictable decrease in body weight (p<0.01) and body fat (p<0.001), both were significantly augmented by the high calcium diets, as shown in FIGS. 9 and 10. Consistent with our previous observations, the use of non-fat dry milk as a calcium source resulted in a significant and substantial amplification of this anti-obesity effect (FIGS. 9 and 10); moreover, fresh whey was as effective as non-fat dry milk in amplifying the anti-obesity effect of calcium, suggesting that most or all of the non-calcium anti-obesity activity of dairy resides in fresh whey.

Increasing calcium intake using a whey-derived mineral mix was not as effective as either the milk or the calcium carbonate in reducing adiposity in Example 1, as it produced little effect on total fat pad mass compared to the low calcium diet. This was suspected to be an anomaly which resulted from the processing of the whey-derived mineral mix, resulting in reduced bioavailability. Consequently, in Example 2, two different sources of whey-derived minerals were used (including another batch of TRUECAL and a batch of VITALARMOR) and found both to produce similar anti-obesity effects which were comparable to, but not greater than, calcium carbonate. Whey-derived mineral preparations of different particle sizes (less than or greater than 10 microns) were compared. This factor was found not to affect the outcome. This indicates that (a) the original finding was indeed an anomaly, and (b) that the “package” of minerals contained in whey is no more effective than isolated calcium in augmenting weight and fat loss. This indicates that non-mineral components of whey must be responsible for the anti-obesity effect. One of these components appears to be whey derived ACE inhibitory activity, as addition of whey-derived ACE inhibitor to the high calcium diet resulted in a further augmentation of fat loss (FIG. 10).

However, although addition of the whey-derived ACE inhibitor to the whey-derived mineral mix significantly increased fat loss, both milk and fresh whey were markedly more effective. This suggests two possibilities. One possibility is that there are additional specific discreet components of whey that provide the additional activity. The other possibility is that although calcium and ACE inhibitor contribute to a portion of the anti-obesity effect of dairy, the overall effect of dairy is likely to result from a synergistic effect among the multiple components of dairy found in fresh whey. If that is the case, it is unlikely that isolated components of dairy are likely to replicate the anti-obesity effect of intact dairy products such as fresh whey protein.

FIG. 11 summarizes the effects of the dietary treatments on skeletal muscle mass. As expected, there was a decline in skeletal muscle mass with energy restriction on the low calcium diet, and addition of calcium (either calcium carbonate or whey-derived mineral mix) with or without ACE inhibition did not modify or attenuate this decrease. In contrast, both the milk and fresh whey-based diets completely prevented the loss of gastrocnemius mass during energy restriction and actually resulted in a modest, but statistically significant (p<0.01) increase compared to the ad libitm fed animals. These data suggest that dairy-based diets promote a repartitioning of dietary energy from adipose tissue to skeletal muscle, resulting in a preservation and/or improvement in lean body mass while adipose tissue is lost.

As set forth in Example 2, intact milk and fresh whey produced the full anti-obesity effect and the skeletal muscle sparing effect. Without intending to be limited by theory, branched chain amino acids in fresh whey and intact milk may contribute to this preservation of skeletal muscle mass.

There are multiple mechanisms whereby calcium and dairy foods may modulate adipocyte function and thereby alter obesity risk. For example, it has been found that increased calcitriol produced in response to low calcium diets stimulates adipocyte [Ca²⁺]ᵢ influx, which results in increased lipogenesis and reduced lipolysis and, consequently, an expansion of adipocyte triglyceride storage. In contrast, higher calcium diets inhibit lipogenesis, promote lipolysis, lipid oxidation and thermogenesis and inhibit diet-induced obesity in mice. In addition, calcitriol inhibits both mitochondrial uncoupling and apoptosis in adipocytes, resulting in increased efficiency of energy storage on low calcium diets, while greater adipocyte uncoupling and apoptosis is found on higher calcium diets. Results of these examples support these conclusions. All high calcium diets suppressed adipocyte intracellular calcium, from 294±46 nM to 118±34 nM on the high calcium diets (p<0.0001), and there was no significant difference among the sources of dietary calcium.

The high calcium diets all suppressed adipose tissue fatty acid synthase gene expression relative to the low calcium diet by 42% (p<0.001), while the fresh whey and milk-based diets resulted in a further suppression of an additional 54% (p<0.001 vs. high calcium). Moreover, addition of the ACE inhibitor to the high calcium diet produced a similar effect (47% further suppression than that found with high calcium; p<0.001 vs. high calcium; not significantly different than milk or fresh whey), suggesting that the additional suppression of fatty acid synthase with dairy vs. calcium is largely due to the presence of ACE inhibitory activity in dairy foods.

The high calcium diets elicited a 63% stimulation of adipose tissue lipolysis (p<0.01) which was augmented by an additional 74% by the fresh whey and milk diets (p<0.01), while the addition of ACE inhibitor to the high calcium diets was without effect. Thus, the additional component(s) of dairy which contribute to higher lipolysis than that found with high calcium diets have even now not been completely identified.

Adipose tissue UCP2 expression was increased ˜2-fold by the high calcium diets (p<0.001), with an additional 35% stimulation on the milk and fresh whey based diets (p<0.01), while the ACE inhibitor was without effect. In skeletal muscle, the high calcium diets were without significant effect on PPARα expression; however, the milk and fresh whey diets elicited a >2-fold stimulation (p<0.01 vs. all other diets), further supporting the role of dairy in re-partitioning available dietary energy from storage in adipose tissue to oxidation in skeletal muscle.

Table 4 summarizes these molecular biology results.

TABLE 4

| Metabolic Indication | Benefit Ranking |
|---|---|
| Intracellular calcium | 3 = 4 = 5 = 6 = 7 = 8 < 2 |
| Adipose tissue fatty acid synthase | 3 = 4 ≈ 8 < 5 = 6 = 7 < 2 |
| Adipose tissue lipolysis | 3 = 4 > 5 = 6 = 7 = 8 > 2 |
| Adipose tissue UCP2 | 3 = 4 > 5 = 6 = 7 = 8 > 2 |
| skeletal muscle PPARα, | 3 = 4 > 5 = 6 = 7 = 8 = 2 |

1 = Ad Lib
2 = loCa
3 = milk
4 = fresh whey protein
5 = casein
6 = soy
7 = fresh whey mineral
8 = fresh whey mineral/ACE

In summary, consuming fresh whey-derived ACE inhibitor provides superior metabolic effects to diets having mineral calcium only or low calcium, at least in terms of adipose tissue fatty acid synthase, and fresh whey and milk provides superior results in terms of the other gene expression measures. These data demonstrate that the additional (non-calcium) anti-obesity of milk resides primarily or exclusively in the whey fraction. Although ACE inhibitory activity of whey contributes to this bioactivity, other factors must act either independently or synergistically to augment it, as the combination of calcium and ACE inhibitor is significantly less effective than milk or fresh whey. Although calcium exerts limited anti-obesity bioactivity in the absence of dairy, it provides no protection against loss of skeletal muscle mass during energy restriction for weight loss, while milk and fresh whey fully protect against this loss and stimulate skeletal muscle PPARα, a marker of lipid oxidation, in this mouse model. ACE inhibition appears to have no role in this; it is proposed that the rich concentration of branched chain amino acids is largely responsible for this effect.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding exemplary specific embodiments are, therefore, to be construed as merely illustrative, and not limitative of the remainder of the disclosure in any way whatsoever. The entire disclosure of all applications, patents and publications, cited above and below and in the figures are hereby incorporated by reference.
1. Zemel, M. B. (2002) Regulation of adiposity and obesity risk by dietary calcium: mechanisms and implications. J Am Coil Nutr 21:146 S-151S
2. Shi, H., Norman, A. W., Okamura, W. H., Zemel, M. B. (2002) 1α, 25-dihydroxyvitamin D₃ inhibits uncoupling protein 2 expression in human adipocytes. FASEB J16:1808-1810 (expanded version on-line: FASEB J 10.1096/fj.02-0255fje, published on-line Sep. 5, 2002)
3. Zemel, M. B. (2002) Calcium, dairy products and weight control. Sciences de Aliments 22:451-458.
4. Zemel, M. B. (2003) Mechanisms of dairy modulation of adiposity. Journal of Nutrition 133:252 S-256S.
5. Sun, X. and Zemel, M. B. (2003) Effects of mitochondrial uncoupling on adipocyte intracellular Ca²⁺ and lipid metabolism. J Nutr Biochem 14:219-226.
6. Zemel, M. B. (2003) The metabolic shift: Role of dietary calcium and dairy products in modulating adiposity. Lipids 38:139-146.
7. Zemel, M. (2003) Calcium modulation of adiposity. Obesity Research 11:375-376.
8. Ha, E., and Zemel, M. B. (2003) Functional properties of whey, whey components, and essential amino acids: Mechanisms underlying health benefits for active people. J Nutr Biochem 14:251-258.
9. Zemel, M. B., Thompson, W., Milstead, A., Morris, K., Campbell, P. (2004) Calcium and dairy acceleration of weight and fat loss during energy restriction in obese adults. Obesity Research 12:582-590.
10. Zemel, M. B. and Miller, S. L. (2004) Dietary calcium and dairy modulation of adiposity and obesity risk. Nutrition Rev 62:125-131.
11. Zemel, M. B. (2004) Role of calcium and dairy products in energy partitioning and weight management. Am J Clin Nutr 79:907s-912s.
12. Sun, X. and Zemel, M. B. (2004) Role of uncoupling protein 2 (UCP2) expression and 1α, 25-Dihydroxyvitamin D3 in modulating adipocyte apoptosis. FASEB J 18:1430-1432.
13. Sun, X. and Zemel, M. B. (2004) Calcium and dairy inhibit weight and fat regain during ad libitum consumption following energy restriction in aP2-agouti transgenic mice. J Nutr 134:3054-3060.
14. Morris, K. L. and Zemel, M. B. (2005) Effect of dietary carbohydrate source on the development of obesity in agouti transgenic mice. Obesity Res (in press)
15. Zemel, M. B., Richards, J., Mathis S., Milstead A., Gebhardt L. and Silva E. (2005). Dairy augmentation of total and central fat loss in obese subjects. Int J Obesity (in press).
16. Zemel, M. B., Zemel, P., Nocton, A-M, Milstead, A., Morris, K. Campbell, P. (2002) Dietary Calcium and Dairy Products Accelerate Weight and Fat Loss During Energy Restriction in Obese Adults. Am J Clin Nutr 75(2S):342S.
17. Zemel, M. B., Morgan, K. (2002) Interaction Between Calcium, Dairy and Dietary Macronutrients in Modulating Body Composition in Obese Mice. FASEB J 301.6.
18. Sun, X., Zemel, M. B. (2002) Effects of Mitochondrial Uncoupling on Adipocyte Intracellular Ca²⁺ and Lipid Metabolism. FASEB J 463.7.
19. Shi, H., Norman, A. W., Okamura, W. H., Zemel, M. B. (2002) 1α,25-Dihydroxyvitamin D₃ (1α,25-(OH)₂-D3) Inhibits Uncoupling Protein 2 Expression in Human Adipocytes. FASEB J 463.8.
20. Zemel, M. B., Nocton, A. M., Richards, J., Milstead, A., Gebhardt, L. Campbell, P. J. (2002) Increasing dairy calcium intake reduces adiposity in obese African-American adults. Circulation 106 (suppl 2) II-610.
21. Zemel, M. B., Nocton, A. M., Richards, J. D., Russell, S. M., Milstead, A. M., Gebhardt, L. P. (2003) Dairy (yogurt) augments fat loss and reduces central adiposity during energy restriction in obese subjects. FASEB J. A1088:679.3.
22. Zemel, M. B., Sobhani, T. (2003) Intracellular calcium modulation of cortisol production in human adipocytes. FASEB J. A323:198.3.
23. Sun X., Zemel, M. B. (2003) Calcium and dairy inhibition of weight and fat regain during ad libitum feeding following energy restriction in aP2-agouti transgenic mice. FASEB J. A746:453.6.
24. Causey, K. R., Zemel, M. B. (2003) Dairy augmentation of the anti-obesity effect of Ca in aP2-agouti transgenic mice. FASEB J. A746:453.7.
25. Truett, G. E., Dubbs, T. M., Urs, S. K., Moustaid-Moussa, N., Zemel, M. B. (2003) Gene expression in inguinal adipose tissue immediately before and after the onset of hyperphagia in fatty rats. FASEB J. A274:177.1.
26. Sun, X., Zemel, M. B. (2003) Role of uncoupling protein 2 (UCP2) expression and 1α,25-Dihydroxyvitamin D₃ in modulating adipocyte apoptosis. Obesity Res. 11:A37.
27. Zemel, M. B., Teegarden, D., Van Loan, M., Schoeller, D. A., Matkovic, V., Lyle R. M., Craig, B. A. (2004) Role of dairy products in modulating weight and fat loss: A multi-center trial. FASEB J 18:abstract 566.5.
28. Melanson, E. L., Ida, T., Donahoo, W. T., Zemel, M. B., Hill, J. O. (2004) The effects of low- and high-dairy calcium diets on resting energy expenditure and substrate oxidation. FASEB J 18:abstract 566.6.
29. Morris, K. L., Miller, S. L., Zemel, M. B. (2004) Calcitriol regulation of 11-∃ hydroxysteroid dehydrogenase 1 (11-∃-HSD1) and angiotensin II receptor 1 (AT1) expresson in human adipocytes. FASEB J 18:abstract 585:11.
30. Radak, T. L., Gertz, E., Zemel, M. B., Teegarden, D., Lyle, R., Craig, B., Matkovic, V., Van Loan, M. D. (2004) Effect of caloric restriction on bone mineral density and bone turnover in overweight individuals with differing calcium intake levels. FASEB J 18:abstract 612.1.
31. Sun, X. and Zemel, M. B. (2004) Reactive oxygen species stimulate cell proliferation and down-regulate UCP expression in 3T3-L1 adipocytes. Obesity Res 12:A21
32. Morris, K. and Zemel, M. B. (2004) Interactive effects of calcitriol and cortisol in regulation of human adipocyte metabolism. Obesity Res 12:A31.

I claim:

1. A method comprising administering to an individual regulating body weight an effective amount of a calcium containing fresh whey product and thereby inducing weight and/or fat loss, preventing weight and/or fat gain, and/or increasing the metabolic consumption of adipose tissue in the individual.

2. The method of claim 1, further comprising maintaining and/or inducing an increase in muscle mass.

3. The method of claim 1, wherein the fresh whey product is as effective as milk for inducing weight and/or fat loss, preventing weight and/or fat gain, increasing the metabolic consumption of adipose tissue in the individual, and/or maintaining muscle mass.

4. The method of claim 1, wherein the individual is maintained on a caloric restricted diet.

5. The method of claim 2, wherein the muscle is maintained in the limbs.

6. The method of claim 2, wherein the muscle is skeletal muscle.

7. The method of claim 1, wherein the administered fresh whey product selectively induces loss of fat pad mass, abdominal fat, perirenal fat or subscapular fat and combinations.

8. The method of claim 1, wherein the fresh whey product induces an increase in muscle mass.

9. The method of claim 1, wherein the fresh whey product comprises whey-derived protein isolate or whey-derived mineral fraction plus whey-derived enzyme-inhibiting peptide fraction, and/or combinations.

10. The method of claim 9, wherein the whey-derived protein isolate comprises bovine serum albumin, alpha-lactoglobulin, beta-lactoglobulin, kappa-casein, lactoferrin, lactoperoxidase, and/or immunoglobulins.

11. The method of claim 9, wherein the whey-derived mineral fraction comprises calcium, copper, magnesium, phosphorus, potassium, selenium, zinc and/or combinations.

12. The method of claim 9, wherein the whey-derived mineral fraction comprises calcium.

13. The method of claim 1, wherein the fresh whey product fortifies a dairy product.

14. The method of claim 14, wherein the dairy product is milk, yogurt or cheese.

15. The method of claim 1, wherein the fresh whey product comprises an enzyme-inhibiting peptide fraction.

16. The method of claim 15, wherein the enzyme-inhibiting peptide comprises angiotensin converting enzyme (ACE) inhibiting peptide.

17. The method of claim 1, wherein the fresh whey product is in the form of a powder.

18. The method of claim 1, wherein the fresh whey product is incorporated into a nutritional or dietary composition or supplement.

19. The method of claim 1, wherein the fresh whey product is incorporated into a food product.

20. The method of claim 19, wherein the food product is a beverage.

21. The method of claim 19, wherein the food product is selected from the group consisting of acidic juice beverages, acidic beverages, neutral pH beverages, nutritional supplement foodstuffs, confectionery products, dairy products, bakery products and farinaceous products.

22. The method of claim 1, wherein the fresh whey product composition is in the form of a tablet, capsule, or combination with other minerals and/or vitamins.

23. The method of claim 1, further comprising administering the fresh whey product to an individual and thereby treating a disorder selected from the group consisting of high blood pressure, stroke, obesity, kidney stones, colon cancer, breast cancer, head and neck tumors, premenstrual syndrome, postpartum depression, hypertensive disorders of pregnancy, Type-2 diabetes, depression, asthma, inflammatory bowel disease, attention deficit disorder, migraine headaches, kidney disease, hypercholesterolemia, congestive heart failure, and immune deficiency.

24. The method of claim 1, wherein the fresh whey product is administered to an individual in need thereof to treat obesity.

25. A method of treating a body weight condition comprising administering to an individual a therapeutically effective amount of a fresh whey product comprising two or more weight control components selected from the group consisting of whey-derived mineral fraction, whey-derived protein isolate, and whey-derived enzyme-inhibiting peptide fraction, thereby inducing weight and/or fat loss, preventing weight and/or fat gain, and/or increasing the metabolic consumption of adipose tissue in the individual.

26. The method of claim 25, further comprising maintaining and/or inducing an increase in muscle mass.

27. A weight management composition comprising therapeutically effective amounts of fresh whey product containing components selected from the group consisting of whey-derived mineral fraction, whey-derived protein isolate and whey-derived enzyme-inhibiting peptide fraction, in a package labeled with information about the effects of consuming effective amounts of the whey product to maintain and/or increase muscle mass while inducing weight and/or fat loss, preventing weight and/or fat gain, and/or increasing the metabolic consumption of adipose tissue in the individual.
